(19) United States
(12) Patent Application Publication
(10) Pub. No.: US 2013/0310625 A1
(43) Pub. Date: Nov. 21, 2013

(54) VELIPARIB IN COMBINATION WITH WHOLE BRAIN RADIATION THERAPY FOR THE TREATMENT OF BRAIN METASTASES

(71) Applicant: AbbVie Inc., North Chicago, IL (US)

(72) Inventor: Vincent L. Giranda, Gurnee, IL (US)

(73) Assignee: ABBVIE INC., North Chicago, IL (US)

(21) Appl. No.: 13/894,555

(22) Filed: May 15, 2013

Related U.S. Application Data
(60) Provisional application No. 61/647,329, filed on May 15, 2012.

(51) Int. Cl. A61N 5/00

(52) U.S. Cl. CPC A61N 5/00
USPC 600/1

(57) ABSTRACT

The invention relates to a method for the treatment of brain metastases from non-small cell lung cancer in a subject, comprising administering to the subject an effective amount of 2-[(2R)-2-methylpyrrolidin-2-yl]-1H-benzimidazole-4-carboxamide, or a pharmaceutically acceptable salt thereof, in combination with an effective amount of whole brain radiation.

### CROSS-REFERENCE TO RELATED APPLICATIONS

This application claims priority to U.S. Provisional Application Ser. No. 61/647,329 filed May 15, 2012, which is incorporated by reference in its entirety.

### FIELD OF THE INVENTION

This invention pertains to the use of veliparib in combination with whole brain radiation therapy (WBRT) for the treatment of patients with brain metastases.

### BACKGROUND OF THE INVENTION

It is estimated that globally over 240,000 people are diagnosed with brain metastases each year. Brain metastases occur in approximately 20-25% of cancer patients with metastases as a result of hematogenous dissemination of systemic cancer. The Incidence may be rising due to better control of systemic disease. More than 50% of the patients will have 1-3 brain metastases at diagnosis. Brain metastases occur most often with lung cancer but also occur frequently in breast, skin (melanoma), kidney, and colon cancers.

In an analysis of Southwest Oncology Group (SWOG) trials, it was found that among 422 patients with non small cell lung cancer (NSCLC), about 64% experienced progressive disease. Among the patients with progressive disease, 26% progressed in the brain (20% brain only and 6% brain plus other site). The median time to brain metastases was approximately 6.5 months, with nearly 25% manifesting brain metastases during initial treatment. (Gasper et al., J Clin Oncol. 2005; 23(13):2955-61).

Symptoms develop rapidly from metastases in the brain compared with other sites because of its rigid encasement, relatively small size of the skull compared with other body cavities, and high sensitivity of the brain to increased intracranial pressure. Brain metastases produce symptoms such as headache, nausea, vomiting, and focal neurological deficit such as motor weakness and focal and generalized seizures in various combinations.

Brain metastases have been shown to result in a poor prognosis for patients with NSCLC. Median survival of between 3 to 4.9 months has been reported for patients with multiple brain metastases from NSCLC. (Horton et al., Am J Roentgenol Radium Ther Nucl Med. 1971; 3:334-35). Nearly one half of patients with brain metastases from NSCLC develop progressive neurologic problems, and only 10% to 15% of patients survive more than 1 year after diagnosis of brain metastases. Survival for patients with up to 2 metastatic organ sites may depend on adequate control of brain metastases.

The management of brain metastases includes both symptomatic and brain-directed therapies. Brain-directed therapies include WBRT, stereotactic radiosurgery (SRS), and neurosurgery as standard of care, either alone or in various combinations, depending on the site, size, and number of brain metastases. None of the currently available treatment modalities has shown a survival advantage against each other in large randomized trials. (Regine et al., Int J Radiat Oncol Biol Phys, 2002; 52(2):333-8). Symptomatic therapy often includes steroid treatment to reduce peri-tumoral edema and anticonvulsants to prevent recurrent seizures. Systemic chemotherapy has limited efficacy in this setting.

For the majority of patients with brain metastases, WBRT is the standard of care. Nonrandomized studies suggest that WBRT increases the median survival time to 3 to 4 months compared to approximately 1 month without treatment and 2 months with steroids alone. Although the response rate of WBRT for brain metastases patients with NSCLC varies, the rate is typically reported to be in the range of 25% to 30%. (Tsao et al., Cochrane Database Syst Rev. 2006; 3:CD003869).

Multiple approaches have been used to improve upon the results of WBRT alone, particularly by adding agents suspected of having radio-sensitizing effects in preclinical models. The following radio-sensitizers have been studied in randomized, controlled studies, all failing to benefit in either local brain tumor control or overall survival: lonidamine, metronidazole, misonidazole, motexafin gadolinium, bromodeoxyuridine, and efaproxiral.

There remains a need for effective methods for the treatment and mitigation of brain metastases and its symptoms. This invention provides methods of treatment that generally address such a need.

### BRIEF SUMMARY OF THE INVENTION

The present invention relates to a method for the treatment of brain metastases from non-small cell lung cancer in a subject, comprising administering to the subject an effective amount of 2-[(2R)-2-methylpyrrolidin-2-yl]-1H-benzimidazole-4-carboxamide (veliparib or ABT-888), or a pharmaceutically acceptable salt thereof, in combination with whole brain radiation therapy.

### BRIEF DESCRIPTION OF THE DRAWINGS

FIG. 1 shows the WBRT Schedule in combination with Veliparib (ABT-888).

FIG. 2 shows Preliminary Mean Pharmacokinetic Parameters of Veliparib following WBRT in Patients with brain metastases.

FIG. 3 shows preliminary mean plasma concentration-time profiles following oral administration of veliparib.

FIG. 4 shows best tumor size percent change from baseline (patients with NSCLC).

FIG. 5 shows best tumor size percent change from baseline (patients with breast cancer).

FIG. 6 shows best tumor size percent change from baseline (patients with other types of cancer).

FIG. 7 shows overall survival for patients with NSCLC (13 events).

FIG. 8 shows overall survival of responders vs. non-responders (patients with NSCLC).

FIG. 9 shows overall survival for patients with breast cancer (13 events).

FIG. 10 shows overall survival of responders vs. non-responders (patients with breast cancer).

FIG. 11 shows observed vs. expected overall survival for patients with NSCLC (20 events).

FIG. 12 shows observed vs. expected overall survival for patients with breast cancer (18 events).

### DETAILED DESCRIPTION OF THE INVENTION

### Definitions

The terms “treat”, “treating” and “treatment” refer to a method of alleviating or abrogating a disease and/or its attendant symptoms.

By “pharmaceutically acceptable” it is meant the carrier, diluent or excipient must be compatible with the other ingredients of the formulation and not deleterious to the recipient thereof.

The “subject” is defined herein to include animals such as mammals, including, but not limited to, primates (e.g., humans), cows, sheep, goats, horses, dogs, cats, rabbits, rats, mice and the like. In preferred embodiments, the subject is a human.

Whole brain radiation therapy (WBRT) is radiation is given to the whole brain, usually over a period of weeks. The radiation is measured in units of gray (Gy), which is the unit of absorbed radiation dose of ionizing radiation.

“Effective amount” or a “pharmaceutically-effective amount” in reference to 2-[(2R)-2-methylpyrrolidin-2-yl]-1H-benzimidazole-4-carboxamide refers to the amount sufficient to induce a desired biological, pharmacological, or therapeutic outcome in a subject.

Brain metastasis or metastases is cancer that has spread from a primary tumor to the brain, i.e., a non-CNS primary solid malignancy.

The present invention provides a method for the treatment of brain metastases from non-small cell lung cancer in a subject, comprising administering to the subject an effective amount of 2-[(2R)-2-methylpyrrolidin-2-yl]-1H-benzimidazole-4-carboxamide, or a pharmaceutically acceptable salt thereof, in combination with an effective amount of whole brain radiation.

In the present invention, the subject has a primary solid malignancy which is non-small cell lung cancer (NSCLC). The NSCLC can be squamous cell carcinoma, adenocarcinoma, or large cell carcinoma. The subject also has brain metastases. In one embodiment, the subject will have at least one lesion. Additionally, the subject may have 2 or more lesions. In one embodiment, the subject has 1 site of metastases from NSCLC, i.e., brain metastases. In another embodiment, the subject has 2 sites of metastases from NSCLC, i.e., brain metastases and metastases in one other organ system.

2-[(2R)-2-methylpyrrolidin-2-yl]-1H-benzimidazole-4-carboxamide is an inhibitor of poly(ADP-ribose)polymerase (PARP) and has been previously described in WO 2006-110816. Poly(ADP-ribose)polymerase has an essential role in facilitating DNA repair, controlling RNA transcription, mediating cell death, and regulating immune response. These actions make PARP inhibitors targets for a broad spectrum of disorders. (Virag L., et al., Pharmacol. Rev. 2002 54(3):375-429). In various preclinical cancer models and human clinical trials, PARP inhibitors have been shown to potentiate radiation and chemotherapy by increasing apoptosis of cancer cells, limiting tumor growth, decreasing metastasis, and prolonging the survival of tumor-bearing subjects. (WO 2007-084532; Donawho C. K., et al., Clin Cancer Res 2007 13(9):2728-37; Kummar S., et al., J Clin Oncol. 2009 27(16):2705-11).

In the treatment of malignant brain tumors, the blood-brain barrier can significantly affect the efficacy of agents. In a preclinical study, 2-[(2R)-2-methylpyrrolidin-2-yl]-1H-benzimidazole-4-carboxamide was shown to cross the blood-brain barrier and accumulate in brain tumor tissue. In addition, in a pre-clinical study with a nonhuman primate model, 2-[(2R)-2-methylpyrrolidin-2-yl]-1H-benzimidazole-4-carboxamide was measured in CSF in a range that has been shown to inhibitor PARP activity in vivo in humans. (Muscal et al. Cancer Chemother Pharmacol. 2010; 65(3):419-25).

This invention also is directed, in part, to all salts of 2-[(2R)-2-methylpyrrolidin-2-yl]-1H-benzimidazole-4-carboxamide and methods of their use. A salt of a compound may be advantageous due to one or more of the salt's properties, such as, for example, enhanced pharmaceutical stability in differing temperatures and humidities, or a desirable solubility in water or other solvents. Where a salt is intended to be administered to a patient (as opposed to, for example, being in use in an in vitro context), the salt preferably is pharmaceutically acceptable and/or physiologically compatible. The term “pharmaceutically acceptable” is used adjectivally in this patent application to mean that the modified noun is appropriate for use as a pharmaceutical product or as a part of a pharmaceutical product. Pharmaceutically acceptable salts include salts commonly used to form alkali metal salts and to form addition salts of free acids or free bases. In general, these salts typically may be prepared by conventional means by reacting, for example, the appropriate acid or base with a compound of the invention.

Pharmaceutically acceptable acid addition salts of 2-[(2R)-2-methylpyrrolidin-2-yl]-1H-benzimidazole-4-carboxamide can be prepared from an inorganic or organic acid. Examples of often suitable inorganic acids include hydrochloric, hydrobromic, hydroiodic, nitric, carbonic, sulfuric, and phosphoric acid. Suitable organic acids generally include, for example, aliphatic, cycloaliphatic, aromatic, araliphatic, heterocyclic, carboxylic, and sulfonic classes of organic acids. Specific examples of often suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), ethanesulfonate, benzenesulfonate, pantothenate, 2-hydroxyethanesulfonate, sulfanilate, cyclohexylaminosulfonate, algenic acid, beta-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, heptanoate, hexanoate, nicotinate, oxalate, palmoate, pectinate, 2-naphthalesulfonate, 3-phenylpropionate, picrate, pivalate, thiocyanate, tosylate, and undecanoate.

Pharmaceutically acceptable base addition salts of 2-[(2R)-2-methylpyrrolidin-2-yl]-1H-benzimidazole-4-carboxamide include, for example, metallic salts and organic salts. Preferred metallic salts include alkali metal (group Ia) salts, alkaline earth metal (group IIa) salts, and other physiologically acceptable metal salts. Such salts may be made from aluminum, calcium, lithium, magnesium, potassium, sodium, and zinc. Preferred organic salts can be made from amines, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups can be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

This invention also is directed, in part, to all compositions of 2-[(2R)-2-methylpyrrolidin-2-yl]-1H-benzimidazole-4-carboxamide and methods of their use. 2-[(2R)-2-methylpyrrolidin-2-yl]-1H-benzimidazole-4-carboxamide may be administered with or without an excipient. Excipients include, but are not limited to, encapsulators and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising 2-[(2R)-2-methylpyrrolidin-2-yl]-1H-benzimidazole-4-carboxamide to be administered orally include, but are not limited to, agar, alginic acid, aluminum hydroxide, benzyl alcohol, benzyl benzoate, 1,3-butylene glycol, carbomers, castor oil, cellulose, cellulose acetate, colloidal silica, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, microcrystalline cellulose, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, titanium dioxide, tragacanth, tetrahydrofurfuryl alcohol, triglycerides, water, mixtures thereof and the like.

Total daily dose of the compositions of the invention to be administered to a human or other mammal host in single or divided doses may be in amounts, for example, from 0.0001 to 300 mg/kg body weight daily and more usually 1 to 300 mg/kg body weight. The dose, from 0.0001 to 300 mg/kg body, may be given twice a day.

In one embodiment of the invention, the dose of 2-[(2R)-2-methylpyrrolidin-2-yl]-1H-benzimidazole-4-carboxamide, or a pharmaceutically acceptable salt or solvate thereof, is in the range of 20 to 600 mg or in the range of 60 to 400 mg. In a further embodiment of the invention, the dose of 2-[(2R)-2-methylpyrrolidin-2-yl]-1H-benzimidazole-4-carboxamide or a pharmaceutically acceptable salt or solvate thereof, is about 30 mg, 50 mg, 80 mg, 100 mg, 150 mg, 200 mg, or 300 mg. The dose can be administered once a day or twice a day. In one embodiment, the dose is administered twice a day.

The present invention further comprises the step of administering whole brain radiation to the subject. Generally, WBRT consists of conventional external beam radiotherapy administered to the whole brain.

WBRT can be administered in any therapeutically effective dose. Typically, WBRT is administered as a course of daily treatment sessions, e.g., fractions. In one embodiment of the invention, the cumulative dose is about 20 Gy to about 40 Gy. In another embodiment, the cumulative dose is about 20 Gy, about 30 Gy, about 35 Gy, or about 37.5 Gy.

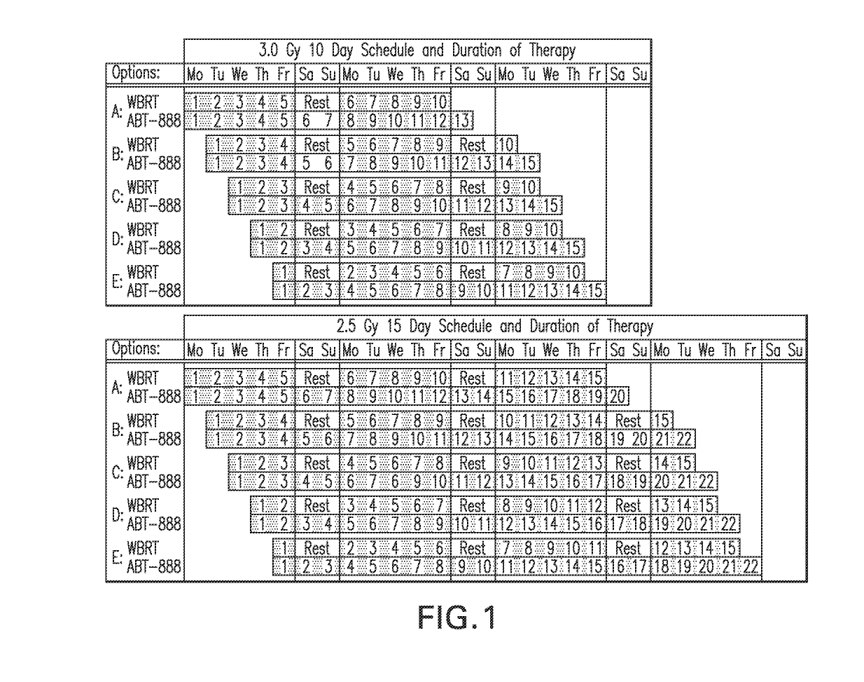

Schedules for the administration of WBRT are presented in FIG. 1.

In one embodiment of the invention, a cumulative dose of 30 Gy is delivered in 3.0 Gy fractions once a day for a total of 10 fractions. In another embodiment, a cumulative dose of 30 Gy is delivered in ten 3.0 Gy fractions over a two week period (Mon-Fri X 2 weeks).

In one embodiment of the invention, a cumulative dose of 37.5 Gy is delivered in 2.5 Gy fractions once a day for a total of 15 fractions. In another embodiment, a cumulative dose of 37.5 Gy is delivered in fifteen 2.5 Gy fractions over a three week period (Mon-Fri X 3 weeks).

In one embodiment of the invention, a cumulative dose of 35 Gy is delivered in 2.5 Gy fractions once a day for a total of 14 fractions.

The administration of 2-[(2R)-2-methylpyrrolidin-2-yl]-1H-benzimidazole-4-carboxamide or a pharmaceutically acceptable salt or solvate thereof, and compositions and formulations thereof, may be prior to, immediately prior to, during, immediately subsequent to or subsequent to the administration of WBRT.

In one embodiment, 2-[(2R)-2-methylpyrrolidin-2-yl]-1H-benzimidazole-4-carboxamide and compositions and formulations thereof are administered on the same day as commencement of WBRT. In another embodiment, 2-[(2R)-2-methylpyrrolidin-2-yl]-1H-benzimidazole-4-carboxamide and compositions and formulations thereof are administered at least one day prior to the commencement of WBRT. In another embodiment, 2-[(2R)-2-methylpyrrolidin-2-yl]-1H-benzimidazole-4-carboxamide and compositions and formulations thereof are administered for two days prior to the commencement of WBRT. In another embodiment, 2-[(2R)-2-methylpyrrolidin-2-yl]-1H-benzimidazole-4-carboxamide and compositions and formulations thereof are administered for one week prior to commencement of WBRT.

In one embodiment, 2-[(2R)-2-methylpyrrolidin-2-yl]-1H-benzimidazole-4-carboxamide and compositions and formulations thereof are administered daily or twice daily continuously during the course of WBRT.

In one embodiment, 2-[(2R)-2-methylpyrrolidin-2-yl]-1H-benzimidazole-4-carboxamide and compositions and formulations thereof are administered subsequent to the completion of a course of WBRT. In another embodiment, 2-[(2R)-2-methylpyrrolidin-2-yl]-1H-benzimidazole-4-carboxamide and compositions and formulations thereof are administered for one day following the last day of a course of WBRT.

In one embodiment, 2-[(2R)-2-methylpyrrolidin-2-yl]-1H-benzimidazole-4-carboxamide and compositions and formulations thereof are administered on days 1-13 while WBRT therapy is administered on days 1-5 and 8-12. In another embodiment, the cumulative dose of WBRT is 30 Gy and 2-[(2R)-2-methylpyrrolidin-2-yl]-1H-benzimidazole-4-carboxamide is administered in a dose of 200 mg twice a day. In yet another embodiment, the cumulative dose of WBRT is 30 Gy and 2-[(2R)-2-methylpyrrolidin-2-yl]-1H-benzimidazole-4-carboxamide is administered in a dose of 50 mg twice a day.

In one embodiment, 2-[(2R)-2-methylpyrrolidin-2-yl]-1H-benzimidazole-4-carboxamide and compositions and formulations thereof are administered on days 1-20 while WBRT therapy is administered on days 1-5 and 8-12 and 15-19. In another embodiment, the cumulative dose of WBRT is 37.5 Gy and 2-[(2R)-2-methylpyrrolidin-2-yl]-1H-benzimidazole-4-carboxamide is administered in a dose of 200 mg twice a day. In yet another embodiment, the cumulative dose of WBRT is 37.5 Gy and 2-[(2R)-2-methylpyrrolidin-2-yl]-1H-benzimidazole-4-carboxamide is administered in a dose of 50 mg twice a day.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

This example is a Phase 1, multicenter, dose-escalation study evaluating the safety, tolerability and pharmacokinetics of veliparib administered concurrently with conventional WBRT in subjects with solid primary tumors metastatic to the brain.

Inclusion Criteria

Patients with histologically or cytologically confirmed non-CNS primary solid malignancy, and pathologically or radiographically confirmed metastatic disease in the brain were eligible for this study. Subjects with non-measurable lesions, including leptomeningeal carcinomatosis were also eligible. WBRT was clinically indicated, with the exception of prophylactic treatment. Patients had Karnofsky Performance Status (KPS) greater than or equal to a score of 70, and adequate hematologic, renal and hepatic function.

Exclusion Criteria

Brain metastases secondary to germ cell tumor or lymphoma malignancy. Primary central nervous system (CNS) neoplasm. Prior or concurrent administration of the following therapies or treatments: (a) prior treatment with WBRT; (b) SRS performed less than 14 days prior to WBRT D1, or is scheduled to occur within 30 days of the last WBRT session; (c) last dose of chemotherapy, immunotherapy, biologic therapy, or investigational therapy, was less than 14 days prior to WBRT D1. Bisphosphonates, hormone modification therapy, and trastuzumab are permitted without restriction. Unresolved or unstable, serious toxicity from prior administration of another investigational drug and/or prior anti-cancer treatment. Known seizure disorder (status epilepticus) that is uncontrolled, or seizures occurring greater than or equal to 3 times a week over the past month. If female, subject is pregnant or breast-feeding. Clinically significant and uncontrolled major cardiac, respiratory, renal, hepatic, gastrointestinal, hematologic or neurological/psychiatric disease or disorder, including but not limited to: (a) active uncontrolled infection; (b) symptomatic congestive heart failure, unstable angina pectoris, or cardiac arrhythmia; and (c) any other illness condition(s) that could exacerbate potential toxicities, confound safety assessments, require excluded therapy for management, or limit compliance with study requirements. Unable to swallow and retain oral medications. Known contraindication to enhanced MRI and CT, including but not limited to: (a) presence of metal objects within the body such as a cardiac pacemaker, implanted cardiac defibrillator, brain aneurysm clips, cochlear implant, ocular foreign body, or shrapnel, and (b) history of immediate or delayed hypersensitivity reaction or other contraindication to contrast agents including but not limited to gadolinium and iodine. Previous enrollment in this study or another study involving the investigation of veliparib, with the exception of receiving a single dose of study drug.

Enrollment

66 patients were dosed from May 2008 to April 2012. 62 patients were off the study treatment. 56 patients completed at least 80% veliparib dose regimen, 58 patients completed the entire course of WBRT and 4 patients are still ongoing.

Baseline characteristics are shown in Table 1.

TABLE 1
Patient and Disease Characteristics

| Baseline Characteristics | All Patients (N = 66, %) |
|---|---|
| Median age, yrs (range) | 58 yrs (31-81) |
| Gender, n (%) | |
| Men/Women | 23 (35%)/43 (65%) |
| KPS, n (%) | |
| 70 | 5 (8%) |
| 80 | 21 (32%) |
| 90 | 25 (38%) |
| 100 | 15 (23%) |
| Prior Anti-Cancer Therapy Agents | |
| Prior therapy agents, n (%) | |
| 0 | 22 (33%) |
| 1-5 | 37 (56%) |
| 6-10 | 5 (8%) |
| >10 | 2 (3%) |
| Prior Radiation Therapy | |
| Yes | 33 (50%) |
| No | 33 (50%) |
| Primary Tumor Type, n (%) | |
| Breast | 21 (32%) |
| Colorectal | 3 (5%) |
| Melanoma | 10 (15%) |
| NSCLC | 23 (35%) |
| Renal | 1 (2%) |
| Other | 8 (12.1) |

Patients received WBRT delivered in 2.5 Gy fractions once a day (QD) for a total of 15 fractions or WBRT delivered at 3.0 Gy fractions QD for a total of 10 fractions.

Veliparib dose-escalation began at 10 mg and increased to 20, 30, 50, 80, 100, 150, 200, and 300 mg twice a day (BID) through out the entire course of WBRT (Table 2); the final WBRT fraction was followed by 1 extra day of veliparib.

| | | | | | Dosing Schedule and Duration of | | |
|---|---|---|---|---|---|---|---|
| | | | | | Therapy | | |
| | | | Daily Dosages | | | Weekends | WBRT |
| | | | ABT-888 | WBRT | WBRT | and | Day 15 |
| Dose Level | | N | (mg) | (Gy) | Days 1 to 15 | Holidays | (+1) |
| Starting | 1 | 3 to 6 | 10 BID = 20 | 2.5 | WBT + ABT- | ABT-888 | ABT-888 |
| Dose | | | | QD | 888 | | |
| | 2 | 3 to 6 | 20 BID = 40 | 2.5 | WBT + ABT- | ABT-888 | ABT-888 |
| | | | | QD | 888 | | |
| | 3 | 3 to 6 | 30 BID = 60 | 2.5 | WBT + ABT- | ABT-888 | ABT-888 |
| | | | | QD | 888 | | |
| | 4 | 3 to 6 | 50 BID = 100 | 2.5 | WBT + ABT- | ABT-888 | ABT-888 |
| | | | | QD | 888 | | |
| | 5 | 3 to 6 | 80 BID = 160 | 2.5 | WBT + ABT- | ABT-888 | ABT-888 |
| | | | | QD | 888 | | |
| | 6 | 3 to 6 | 100 BID = 200 | 2.5 | WBT + ABT- | ABT-888 | ABT-888 |
| | | | | QD | 888 | | |
| | 7 | 3 to 6 | 150 BID = 300 | 2.5 | WBT + ABT- | ABT-888 | ABT-888 |
| | | | | QD | 888 | | |
| | 8 | 3 to 6 | 200 BID = 400 | 2.5 | WBT + ABT- | ABT-888 | ABT-888 |
| | | | | QD | 888 | | |
| | 9 | 3 to 6 | 250 BID = 500 | 2.5 | WBT + ABT- | ABT-888 | ABT-888 |
| | | | | QD | 888 | | |
| | 10 | 3 to 6 | 300 BID = 600 | 2.5 | WBT + ABT- | ABT-888 | ABT-888 |
| | | | | QD | 888 | | |
| | 11 | 3 to 6 | 350 BID = 700 | 2.5 | WBT + ABT- | ABT-888 | ABT-888 |
| | | | | QD | 888 | | |
| | 12 | 3 to 6 | 400 BID = 800 | 2.5 | WBT + ABT- | ABT-888 | ABT-888 |
| | | | | QD | 888 | | |

| | | | |
|---|---|---|---|
| MTD and/or RPTD 2.5 QD | WBT + ABT- | ABT-888 | ABT-888 |
| | 888 | | |
| MTD and/or RPTD 3.0 QD | WBT + ABT- | ABT-888 | ABT-888* |
| | 888* | | |

*WBRT (3.0 Gy) will be administered on Days 1 to 10, excluding weekends and holidays, with continuous dosing of ABT-888 on Days 1 to 10 and Day 10 (+1).

PK, Safety, and tumor response by RECIST were assessed.

PK

To determine PK of veliparib, intensive PK sampling was performed on Day 1 of the WBRT schedules (15 and 10 day) at the following time points: 0, 30, 1, 2, 6 and 24 hours. PK as was also performed on Day 6 for both WBRT schedules at the following time points: 0, 30 min, 1, 2, and 6 For the 15 Day schedule sampling was performed on Day 15 at 0, 30 min, 1, 2 and 6 hours. For the 10 day schedule only a 0 hour sample was collected. Post treatment samples were collected 24-72 hours after the last dose of veliparib for both WBRT schedules.

Veliparib was rapidly absorbed with an average Tₘₐₓ value of 1 to 2 hours

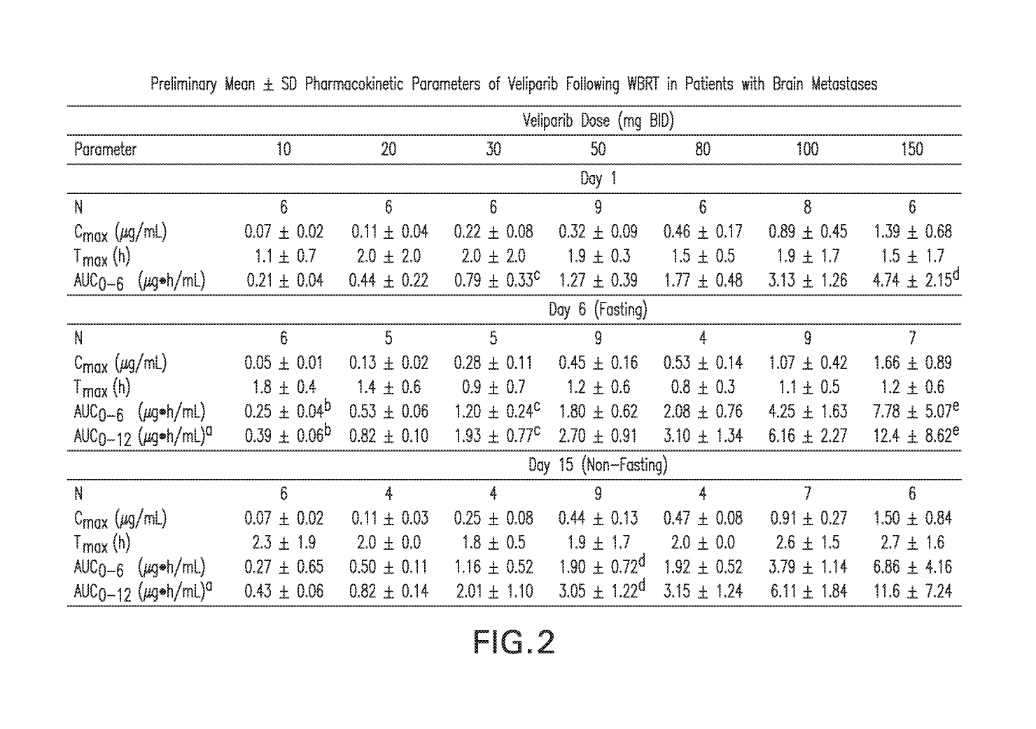

Increase in veliparib exposure (Cₘₐₓ and AUC) with increase in veliparib dose was approximately dose-proportional over 10 to 150 mg BID dose range, with an oral clearance of 21.6±14.2 L/h (mean±SD, n=45; FIG. 2)

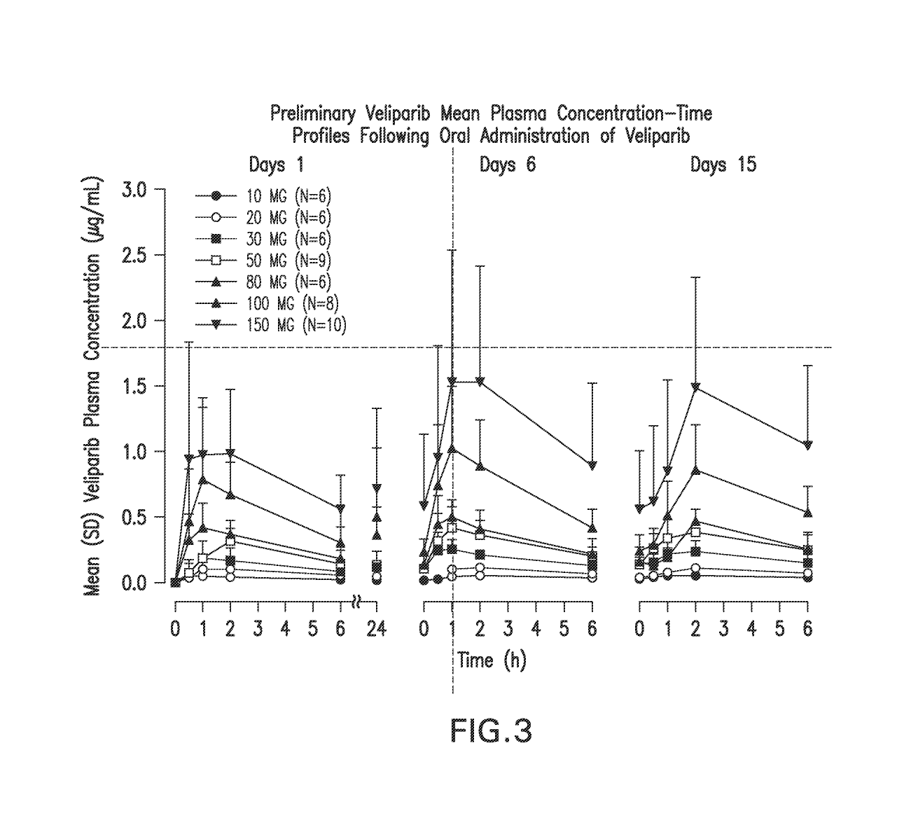

Comparison of Veliparib Cₘₐₓ and AUC values on Day 1, Day 6 and Day 15 demonstrate that veliparib accumulation following BID dosing is minimal (FIG. 3)

Veliparib Exposure on Day 6 (fasting) and Day 15 (non-fasting) were comparable indicating no significant effect of food on veliparib oral bioavailability

Safety

AEs were assessed by NCI CTCAE v3.0.

The most common (≧10%) treatment-emergent AEs that were possibility or probably related to veliparib are shown in Table 3.

TABLE 3
Treatment-Emergent All Grade AEs Possibly or Probably Related
to Veliparib Occurring in ≧10% of Patients and
All Grade 3/4 AEs; N = 66

| | Any Grade | Grade 3/4 |
|---|---|---|
| Events | n (%) | n (%) |
| Any Event | 37 (56) | 10 (15) |
| Fatigue | 22 (33.3) | 3 (5) |
| Nausea | 13 (19.7) | 1 (2) |
| Vomiting | 8 (12) | — |
| Decreased Appetite | 8 (12) | — |

No dose limiting toxicities (DLTs) were observed at the 10, 20, 30, 50, 80, 100, 200 and 300 mg dose levels.

At 150 mg veliparib BID, 1 patient experienced a DLT of grade 3 hypokalemia and a second patient experienced a DLT of grade 3 hyponatraemia. 8 (12%) patients had AEs leading to study drug discontinuation. 2 (3%) patients had AEs leading to study drug discontinuation possibly or probably related to veliparib.

Efficacy

Survival follow-up assessments begin post the final visit at 3 month intervals up to 2 years and tumor assessments and mini-mental state examinations are performed at each follow-up visit. Radiographic assessments of the brain using cranial gadolinium-enhanced MRI were performed for all patients at baseline, final visit and at every 3 months interval after WBRT. Disease response/progression was assessed using RECIST as per investigator assessment.

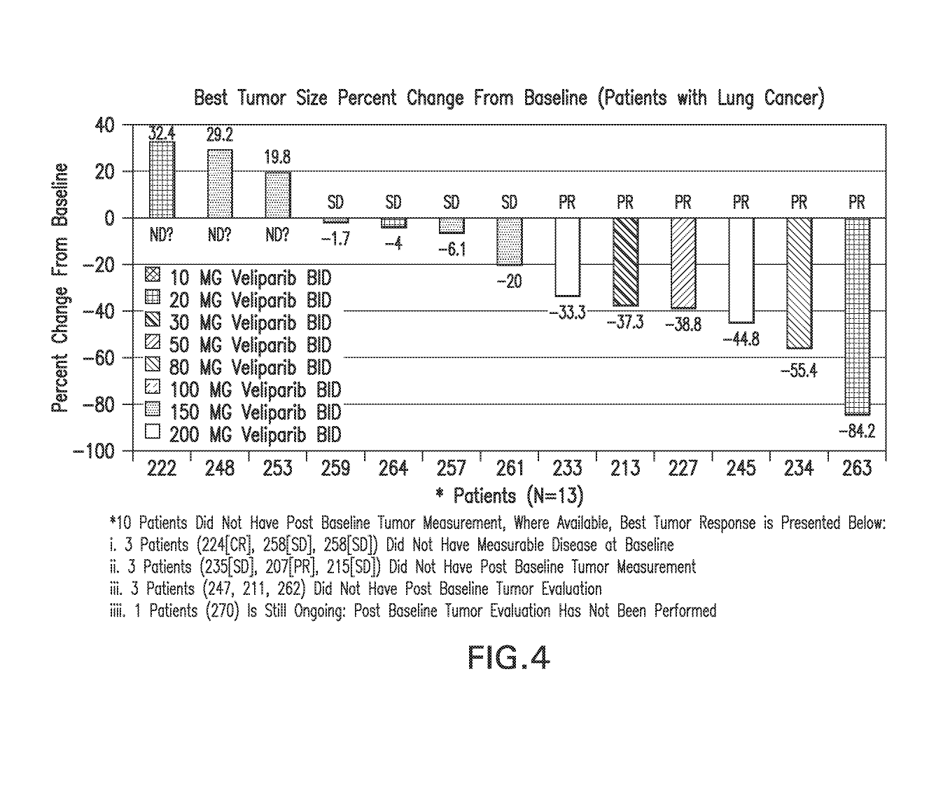

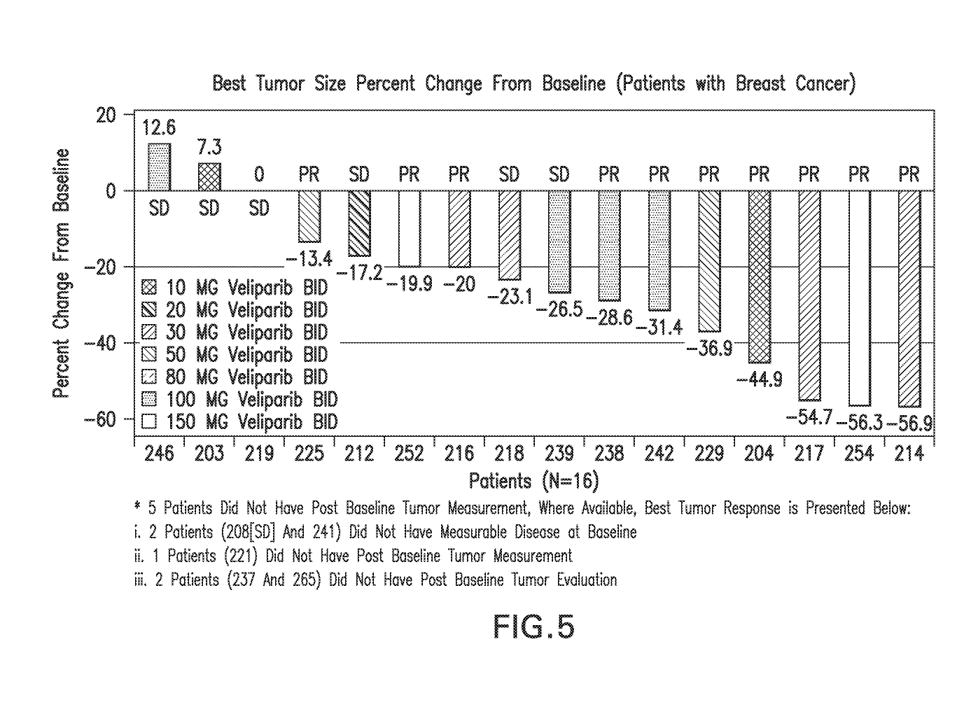

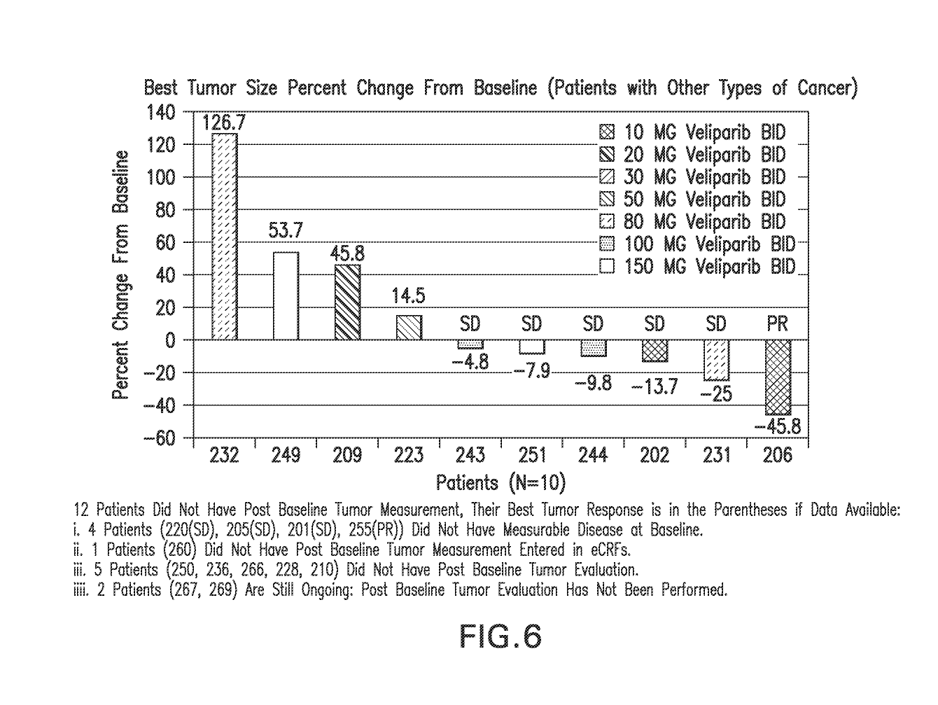

Of the 66 patients dosed, 54 had measurable disease at baseline. Best brain tumor response data are presented for patients with NSCLC (FIG. 4), Breast cancer (FIG. 5) and other tumor types (FIG. 6).

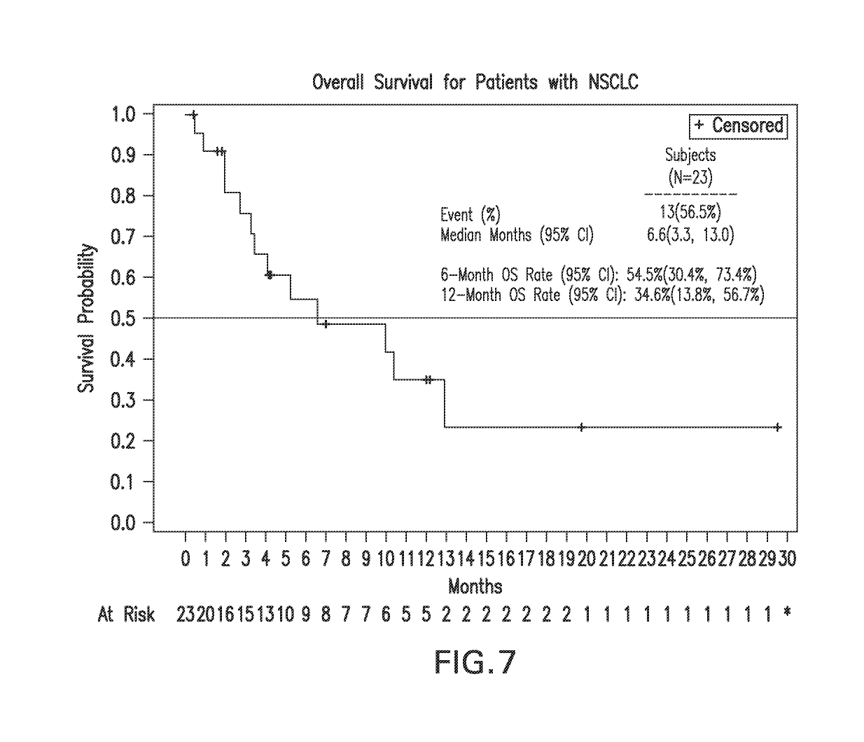

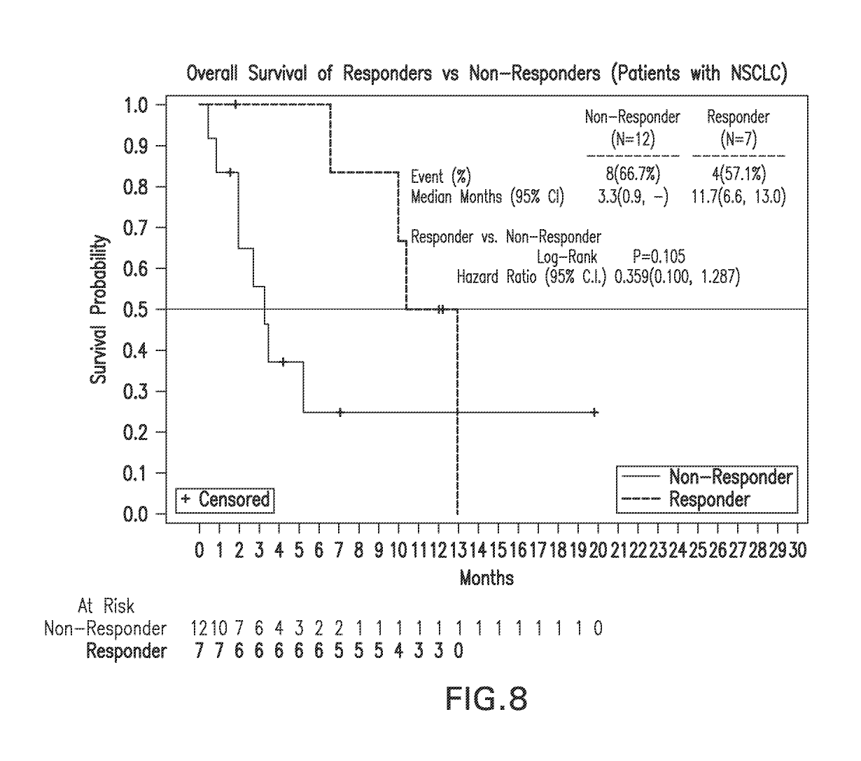

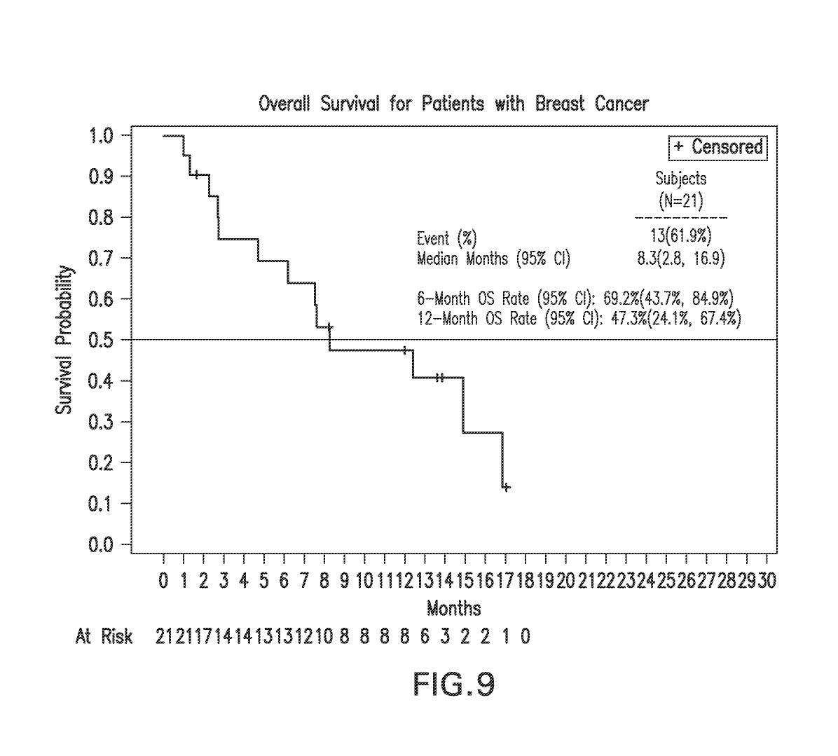

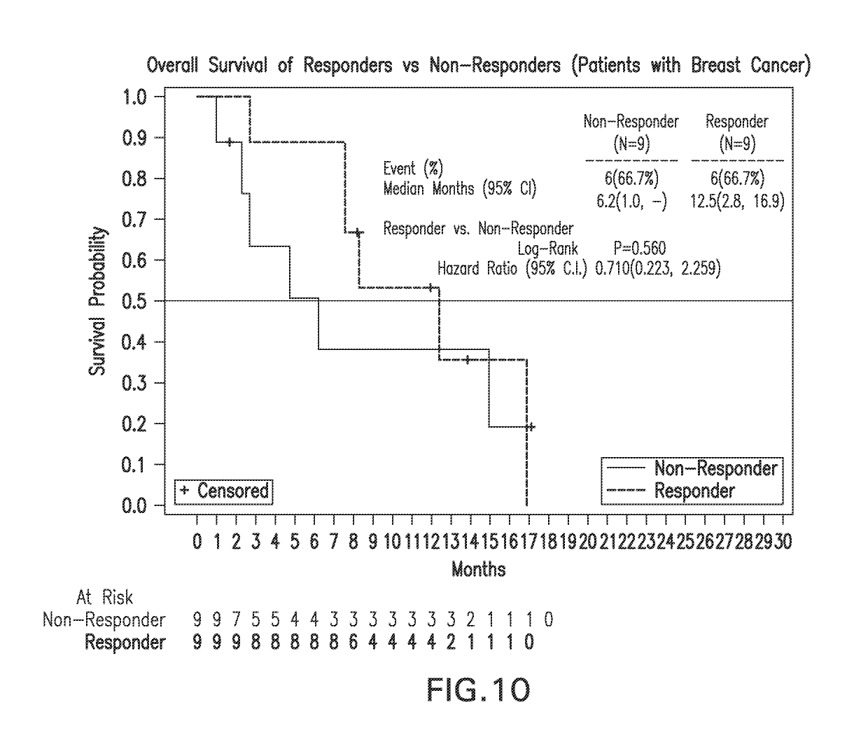

In addition to median survival time estimates for NSCLC patients (FIG. 7) and breast cancer patients (FIG. 9), median survival time was also estimated for responders vs non-responder in patients with NSCLC (FIG. 8) and Breast cancer (FIG. 10).

By March 2013, a total of 81 patients were enrolled. Final baseline characteristics are shown in Table 4.

TABLE 4
Final Patient and Disease Characteristics

| Baseline Characteristics | All Patients (N = 81, %) |
|---|---|
| Median age, yrs (range) | 58 yrs (31-84) |
| Gender, n (%) | |
| Men/Women | 26 (32%)/55 (68%) |
| KPS, n (%) | |
| 70 | 9 (11.1%) |
| 80 | 29 (35.8%) |
| 90 | 28 (34.6%) |
| 100 | 15 (18.5%) |
| Prior Anti-Cancer Therapy Agents | |
| Prior therapy agents, n (%) | |
| 0 | 26 (32.1%) |
| 1-5 | 45 (55.6%) |
| 6-10 | 8 (9.9%) |
| >10 | 2 (2.5%) |
| Prior Radiation Therapy | |
| Yes | 41 (50.6%) |
| No | 40 (49.4%) |
| Primary Tumor Type, n (%) | |
| Breast | 25 (30.9%) |
| Colorectal | 2 (2.5%) |
| Melanoma | 11 (13.6%) |
| NSCLC | 34 (42%) |
| Renal | 1 (1.2%) |
| Other | 7 (8.6%) |
| Missing | 1 (1.2%) |

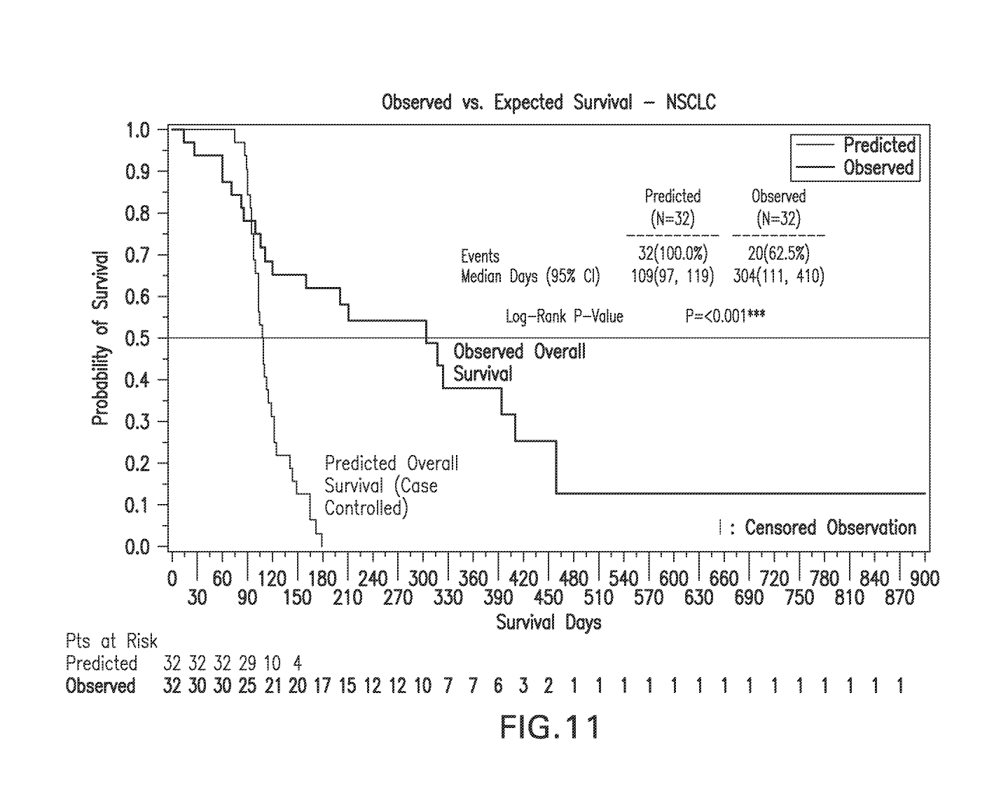

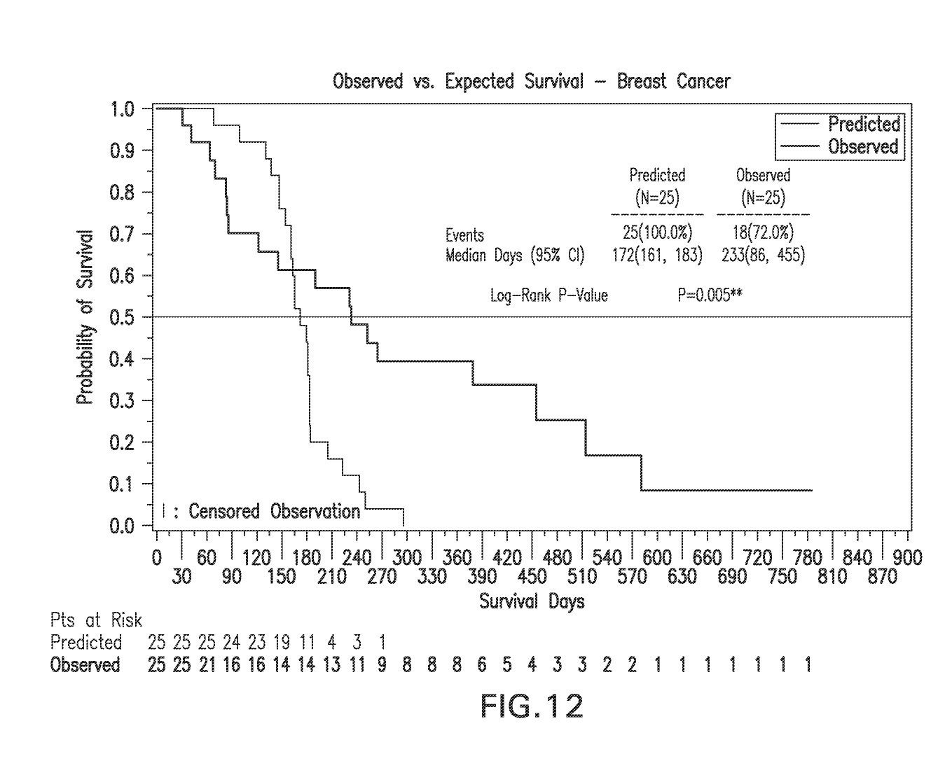

Observed overall survival is plotted for NSCLC patients (FIG. 11) and breast cancer patients (FIG. 12) vs. Expected survival. Expected survival was estimated using a nomogram for individualized prediction of survival for brain metastasis patients (see Neuro-Oncology, 2012).

What is claimed is:

1. A method for the treatment of brain metastases from non-small cell lung cancer in a subject, comprising administering to the subject an effective amount of 2-[(2R)-2-methylpyrrolidin-2-yl]-1H-benzimidazole-4-carboxamide, or a pharmaceutically acceptable salt thereof, in combination with an effective amount of whole brain radiation.

2. The method of claim 1, wherein the amount of 2-[(2R)-2-methylpyrrolidin-2-yl]-1H-benzimidazole-4-carboxamide is from about 30 to about 600 mg per day.

3. The method of claim 1 or 2, wherein the amount of 2-[(2R)-2-methylpyrrolidin-2-yl]-1H-benzimidazole-4-carboxamide is from about 60 to about 400 mg per day.

4. The method of any of claims 1-3, wherein the amount of 2-[(2R)-2-methylpyrrolidin-2-yl]-1H-benzimidazole-4-carboxamide is about 100 mg per day.

5. The method of any of claims 1-3, wherein the amount of 2-[(2R)-2-methylpyrrolidin-2-yl]-1H-benzimidazole-4-carboxamide is about 400 mg per day.

6. The method of any of claims 1-5, wherein the 2-[(2R)-2-methylpyrrolidin-2-yl]-1H-benzimidazole-4-carboxamide is administered twice a day.

7. The method of any of claims 1-6, wherein the amount of whole brain radiation is a cumulative dose of about 20 Gy to about 40 Gy.

8. The method of claim 7, wherein the cumulative dose is about 30 Gy.

9. The method of claim 8, wherein the cumulative dose was delivered in about 3.0 Gy fractions once a day for a total of 10 fractions.

10. The method of claim 7, wherein the cumulative dose is about 37.5 Gy.

11. The method of claim 10, wherein the cumulative dose was delivered in about 2.5 Gy fractions once a day for a total of 15 fractions.

12. The method of claim 7, wherein the cumulative dose is about 35 Gy.

13. The method of claim 12, wherein the cumulative dose was delivered in about 2.5 Gy fractions once a day for a total of 14 fractions.

14. The method of any of claims 1-13, wherein the 2-[(2R)-2-methylpyrrolidin-2-yl]-1H-benzimidazole-4-carboxamide is administered before administration of the whole brain radiation therapy.

15. The method of any of claims 1-13, wherein the 2-[(2R)-2-methylpyrrolidin-2-yl]-1H-benzimidazole-4-carboxamide is administered concurrently with administration of the whole brain radiation therapy.

16. The method of any of claims 1-13, wherein the 2-[(2R)-2-methylpyrrolidin-2-yl]-1H-benzimidazole-4-carboxamide is administered after administration of the whole brain radiation therapy.
